(19) United States
(12) Patent Application Publication
(10) Pub. No.: US 2020/0030264 A1
(43) Pub. Date: Jan. 30, 2020

(54) TOPICAL ANESTHETIC FOR USE WITH LASER RADIATION AND METHODS OF FORMING SAME

(71) Applicant: COLLABORATIVE OPERATION PHARMACISTS TRADING, Spring, TX (US)

(72) Inventor: Markou Gabir, Spring, TX (US)

(21) Appl. No.: 16/522,573

(22) Filed: Jul. 25, 2019

Related U.S. Application Data
(60) Provisional application No. 62/711,661, filed on Jul. 30, 2018.

(51) Int. Cl. A61K 31/167; A61K 31/245; A61K 47/38; A61K 9/00; A61K 9/06

(52) U.S. Cl. CPC A61K 31/167; A61K 31/245; A61K 9/06; A61K 9/0014; A61K 9/0034; A61K 47/38

(57) ABSTRACT

A pharmaceutical composition for topical administration includes a therapeutically safe and effective amount of a lidocaine salt, a therapeutically safe and effective amount of a tetracaine salt, distilled water, and hydroxyethyl cellulose.

### CROSS-REFERENCE TO RELATED APPLICATIONS

The present application claims priority to U.S. Provisional Patent Application Ser. No. 62/711,661 filed Jul. 30, 2018, the contents of which are hereby incorporated in their entirety as if fully set forth herein.

### FIELD OF THE DISCLOSURE

The present invention relates to the field of anesthetics, and more in particular the field of topical anesthetics for use with laser radiation.

### BACKGROUND OF THE INVENTION

Multiple applications of laser radiation are known for surgical, aesthetic or therapeutic treatment of the human body. In some applications, laser is used as a cutting instrument in replacement of scalpels. In other applications, laser is used for causing the necrosis of neoplastic tissues, for the bio-stimulation of the growth of particular types of tissues, for example of the cartilaginous tissue, for pain treatment. In applications more strictly related to aesthetic treatments, laser radiation is used for wrinkle reduction, skin rejuvenation, hair scalp treatment for favoring hair growth, stimulation of collagen production, etc.

Recently, lasers have been used to treat the vaginal canal by a laser beam, comprising a vaginal canal wall retractor, associated to a system for directing the laser beam towards the wall. This allows using the laser beam for treating the mucosa of the vaginal canal to prevent and treat atrophic vaginitis, a condition typical but not exclusive of the post-menopause period that currently is normally treated with estrogens for short periods. Atrophic vaginitis is a pathological condition characterized by an inflammation of the vaginal mucosa with progressive decrease of the mucosa thickness due to the loss of collagen structure. Atrophic vaginitis is a highly disabling condition responsible for considerable psychological discomfort for women suffering from this condition, due to the associated pain, burn, bleeding, ectropion and due to the impossibility of having normal sexual intercourse (dyspareunia). Other conditions may require similar laser radiation treatments, including microblading, and others.

The user of laser radiation as part of the treatment regimen may be painful or cause some discomfort to the patient. Traditional topical anesthetics may reduce the effectiveness of the laser radiation treatment. Thus, it would be beneficial to provide a topical anesthetic for use with similar laser radiation treatments that provides relief to the patient without reducing the efficacy of the laser treatment.

### SUMMARY OF THE INVENTION

In some embodiments, a pharmaceutical composition for topical administration includes a therapeutically safe and effective amount of a lidocaine salt, a therapeutically safe and effective amount of a tetracaine salt, distilled water, and. hydroxyethyl cellulose.

In some embodiments a method of treating tissue includes administering pharmaceutical composition to the tissue, the composition including a therapeutically safe and effective amount of a lidocaine salt, a therapeutically safe and effective amount of a tetracaine salt, distilled water, and hydroxyethyl cellulose, and treating the tissue with laser energy.

### DETAILED DESCRIPTION OF AN EMBODIMENT OF THE INVENTION

Atrophic vaginitis and other similar conditions may be prevented or treated by impinging the mucosa that covers the vaginal canal through a preferably pulsed laser beam of suitable wavelength and power, which allows renewing the epithelium, that is, the mucosa surface layer.

One example of a laser suitable for performing such procedures is the diVa laser treatment developed by SCITON®, which provides a hybrid fractional laser capable of generating ablative and non-ablative wavelengths.

In some examples, a laser source may include a pulsed source, for example with pulses with an emission lasting between 0.1 and 10 milliseconds and preferably between 0.2 and 2 milliseconds, or a continuous source with emission times between 0.5 and 50 milliseconds. The laser radiation may have a wavelength comprised for example between 1,000 nm and 12,000 nm, preferably it may be equal to 10,600 nm.

The power of the beam emitted by the source may be selected so that the beam has the effect of epithelium renewal and stimulation of collagen production on the mucosa. Typically, the power may be comprised between 2 and 100 W, preferably between 10 and 50 W.

The apparatus may comprise systems for controlling the scanning mirrors, so as to move the pulsed or continuous beam for performing a treatment according to a process that provides for impinging the mucosa with laser pulses in zones or points adjacent to one another and consecutive along a predetermined path, wherein the spacing between the scanning points may preferably be comprised between 0 and 5,000 micrometers, and preferably between 50 and 5,000 micrometers and even more preferably between 200 and 2,000 micrometers.

To reduce pain or discomfort to the patient, a topical anesthetic may be provided for use with such laser radiation treatments. In one example, the present disclosure includes a topical anesthetic formed of a safe and effective amount of a pharmaceutically acceptable lidocaine salt and/or a pharmaceutically acceptable tetracaine salt. In some examples, a composition includes an amount of lidocaine hydrochloride of between 4% and 8% w/w. Preferably, the anesthetic includes 8% w/w of lidocaine hydrochloride. The anesthetic may further include a safe and effective amount of a tetracaine salt gel in addition to the lidocaine salt or instead of it. In some examples, a tetracaine hydrochloride gel is used of between 4% and 8% w/w, and preferably approximately 6% w/w. By using lidocaine hydrochloride and a tetracaine hydrochloride gel, a composition may be formed that is clear, which provides the anesthetic benefits without reducing the performance of the laser therapy. In at least some examples, the composition may have a sufficient clarity of. In at least some examples, the composition is at least partially translucent or transparent such that a predetermined amount of light can pass through it. Alternatively, the composition may have the same clarity as glycerol.

The composition may further include inactive ingredients of distilled water and hydroxyethyl cellulose to provide a viscosity equal to or similar to glycerol. In at least some examples, the resulting composition may have a viscosity of between 1.3 and 1.5 Pa·s. In at least some examples, the composition may have a viscosity of 1.412 Pa·s.

In at least some examples, the composition is formed by combining 16 grams of lidocaine hydrochloride, 12 grams tetracaine hydrochloride gel, 200 mL of distilled water and 3.133 grams of hydroxyethylcellulose powder.

The therapeutic efficacy of a topical gel formulation also depends upon its spreadability as spreadability determines the ease of the composition's application. Specifically, spreadability is the term expressed to denote the extent of area over which the formulation readily spreads upon application to skin or in the affected part. Thus, it also affects the therapeutic benefit derived from the formulation. Compositions formed as described in the instant disclosure had desirable spreadability characteristics. Additionally, one of the most important properties of a mucoadhesive polymer is the viscosity of the aqueous solution and the instant compositions provided desirable results.

The composition may be packaged in pre-filled syringes, each syringe having approximately 3-4 mL. In use, a physician may apply the topical anesthetic to the patient's skin before beginning the laser treatment. The composition's clarity and viscosity may allow it to provide effective pain relief without reducing the effectiveness of the laser treatment.

The term “safe and effective amount” as used herein, means an amount of an active ingredient or a composition high enough to deliver the desired benefit, but low enough to avoid serious side effects within the scope of medical judgment. The term “pharmaceutically acceptable” as used herein, means suitable for use in contact with the tissues without undue toxicity, irritation, incompatibility, instability, allergic response, and the like. All numerical ranges used herein, are meant to be inclusive of their upper and lower limits. Unless noted otherwise, all percentages in the compositions of the invention are on a weight-to-weight basis. Throughout the description and claims the word “comprise” is not intended to exclude other technical features, additives, components, or steps. Additional objects, advantages and features of the invention will become apparent to those skilled in the art upon examination of the description or may be learned by practice of the invention. The following particular embodiments are provided by way of illustration, and is not intended to be limiting of the present invention. it is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection individual embodiments may be shared with others of the described embodiments.

1. A pharmaceutical composition for topical administration comprising:
a therapeutically safe and effective amount of a lidocaine salt;
a therapeutically safe and effective amount of a tetracaine salt;
distilled water; and
hydroxyethyl cellulose.

2. The composition of claim 1, wherein the lidocaine salt includes lidocaine hydrochloride.

3. The composition of claim 1, wherein the lidocaine salt includes lidocaine hydrochloride in an amount of between about 7% and about 8% w/w.

4. The composition of claim 1, wherein the tetracaine salt includes tetracaine hydrochloride.

5. The composition of claim 1, wherein the tetracaine salt includes tetracaine hydrochloride in an amount of between about 5% and about 7% w/w.

6. The composition of claim 1, wherein the composition is substantially clear.

7. The composition of claim 1, wherein the composition has a viscosity similar to that of glycerol.

8. A method of treating tissue comprising:
administering a pharmaceutical composition to the tissue, the composition including a therapeutically safe and effective amount of a lidocaine salt, a therapeutically safe and effective amount of a tetracaine salt, distilled water, and hydroxyethyl cellulose; and
treating the tissue with laser energy.

9. The method of claim 8, wherein treating the tissue with laser energy includes applying at least one of an ablative wavelength and a non-ablative wavelength to the tissue.

10. The method of claim 9, wherein treating the tissue with laser energy includes applying both an ablative wavelength and a non-ablative wavelength to the tissue.

11. The method of claim 9, wherein administering a pharmaceutical composition to the tissue includes administering a clear pharmaceutical composition.

12. The method of claim 9, wherein administering a pharmaceutical composition to the tissue includes administering a viscous pharmaceutical composition having a viscosity of at least 1.3 Pa·s.

13. The method of claim 9, wherein administering a pharmaceutical composition to the tissue includes administering a composition having lidocaine hydrochloride.

14. The method of claim 9, wherein administering a pharmaceutical composition to the tissue includes administering a composition having lidocaine hydrochloride in an amount of between about 7% and about 8% w/w.

15. The method of claim 9, wherein administering a pharmaceutical composition to the tissue includes administering a composition having tetracaine hydrochloride.

16. The method of claim 9, wherein administering a pharmaceutical composition to the tissue includes administering a composition having tetracaine hydrochloride in an amount of between about 5% and about 7% w/w.
